(19) United States
(12) Patent Application Publication
(10) Pub. No.: US 2020/0409978 A1
(43) Pub. Date: Dec. 31, 2020

(54) Apparatus and Method for Assessment of Patient Condition

(71) Applicant: Modernizing Medicine, Inc., Boca Raton, FL (US)

(72) Inventors: Daniel Cane, Boynton Beach, FL (US); Michael Sherling, Boca Raton, FL (US)

(73) Assignee: Modernizing Medicine, Inc., Boca Raton, FL (US)

(21) Appl. No.: 16/849,141

(22) Filed: Apr. 15, 2020

Related U.S. Application Data
(63) Continuation of application No. 16/109,878, filed on Aug. 23, 2018, now abandoned.
Application No. 16/109,878 is a continuation of application No. 15/168,444, filed on May 31, 2016, now abandoned.
Application No. 15/168,444 is a continuation of application No. 14/193,050, filed on Feb. 28, 2014, now abandoned.
(60) Provisional application No. 61/771,538, filed on Mar. 1, 2013.
(60) Provisional application No. 61/793,378, filed on Mar. 15, 2013.

(51) Int. Cl. G06F 16/28; G06N 20/00; G16H 40/63; G16H 40/20; G16H 50/70; G16H 50/20

(52) U.S. Cl. CPC G06F 16/283; G06N 20/00; G16H 40/63; G16H 80/00; G16H 50/70; G16H 50/20; G16H 40/20

(57) ABSTRACT

A medical information system. In one embodiment, the medical information system includes: a learning engine; a user interface in communication with the learning engine; and a data warehouse in communication with the learning engine, wherein the learning engine will generate a report on a patient in response to patient data. In another embodiment, the data warehouse includes at least one data mart comprising summarized and indexed aggregated data that are pre-calculated and pre-joined. In one embodiment, the learning engine filters requests for aggregated data based on parameters supplied by the user interface. In another embodiment, a user may query medicine treatment statistics in response to diagnostics. In yet another embodiment, some of the plurality of preferences of the physician are predetermined by selection by the physician. In still yet another embodiment, some of the plurality of preferences of the physician are predetermined by actions taken by the physician.

### RELATED APPLICATIONS

This application claims priority to U.S. Provisional Application No. 61/771,538, filed Mar. 1, 2013, and U.S. Provisional Application No. 61/793,378, filed Mar. 15, 2013, the contents of each of which are herein incorporated by reference.

### FIELD OF THE INVENTION

The invention relates generally to the field of medical data and more specifically to the field of medical diagnosis and data display.

### BACKGROUND OF THE INVENTION

Studies have shown that physicians spend 45% of their time outside of the examination room on such matters as filling out paperwork for patient notes and billing documentation. Even computer-literate clinicians take four minutes to record a note using standard template-based electronic medical records software. As a result, the act of completing a patient record is time consuming.

Such record keeping also has an economic aspect. Payment depends in part on record keeping. The physician must confirm that the correct treatment codes are entered, and that various other required documentation is in place. Because of these complicated billing requirements, some physicians, concerned that mistakes in billing entries might lead to an audit, under-code or claim to have done less work than they actually performed to avoid missing something in the required documentation. Conversely, physicians may forget to enter key components of documentation which the Centers for Medicare and Medicaid Services (“CMS”) treats as an over-code. As far as CMS is concerned, if the documentation is not correct, the examination or procedure did not occur.

Most importantly, the complexity of medicine may mean that a physician may not be taking advantage of the latest information available to treat patients. The physician may not be aware of the information, or the information may have slipped his or her mind. Furthermore, the variety of medical tests and results that are available in most patients' medical histories means that for any given patient, trends and treatments may not be easy to discern.

To address these issues, a number of template-based software systems exist into which physicians enter data on the computer. In general, not only do these systems barely reduce the time it takes for a physician to enter a note into the medical record of a patient, but they do not provide any additional information to aid the physician in treating the patient.

Thus, the issue is that current note taking and diagnosis session-related documentation by a physician using a computer is time consuming, fraught with errors, inefficient, and insufficient.

What is needed is an intelligent system that will enter data with minimal physician interaction. The present invention addresses this need.

### SUMMARY OF THE INVENTION

In one aspect, the invention relates to a medical information system. In one embodiment, the medical information system includes: a learning engine; a user interface in communication with the learning engine; and a data warehouse in communication with the learning engine wherein the learning engine will generate any of a number of reports relating to a current patient based in response to the current patient's diagnostic data, and current patient's demographics and patient data and demographics of other patients' data in the warehouse. In another embodiment, the data warehouse includes at least one data mart comprising summarized and indexed aggregated data that are pre-calculated and pre-joined. In yet another embodiment, the generated report comprises one or more of a measure of the popularity and a measure of efficacy of treatment and a determination of the amount of reimbursement paid on billing. In yet another embodiment, the diagnoses are aggregated by their ICD codes. In still yet another embodiment, medicines are aggregated by their FDB/RxNorm drug name.

In one embodiment, the learning engine will filter requests for aggregated data based on parameters supplied by the user interface. In another embodiment, a user may query medicine treatment statistics in response to diagnostics. In yet another embodiment, some of the plurality of preferences of the physician are predetermined by selection by the physician. In still yet another embodiment, some of the plurality of preferences of the physician are predetermined by actions taken by the physician.

In one embodiment, the learning engine further comprises an interactive 3-D body atlas comprising a plurality of tissue levels, wherein the physician may annotate the body atlas with patient data. In another embodiment, the annotated body atlas with patient data is linked to other aggregated data in the database. In yet another embodiment, the learning engine will generate a report on a patient based in response to a plurality of preferences of the physician attending to the patient. In still yet another embodiment, patient trend data is displayed on the user interface.

In another aspect, the invention relates to a method of using a medical information system comprising: a learning engine; a user interface in communication with the learning engine; and a data warehouse in communication with the learning engine. In one embodiment, the method comprises the step of generating a report on a patient by the learning engine based in response to patient data. In another embodiment, the method further includes the step of summarizing and indexing aggregated data that are pre-calculated and pre-joined in at least one data mart. In yet another embodiment, the method includes the step of filtering requests for aggregated data based on parameters supplied by the user interface.

### BRIEF DESCRIPTION OF THE DRAWINGS

The structure and function of the invention can be best understood from the description herein in conjunction with the accompanying figures. The figures are not necessarily to scale, emphasis instead generally being placed upon illustrative principles. The figures are to be considered illustrative in all aspects and are not intended to limit the invention, the scope of which is defined only by the claims.

FIG. 1(a) is a diagram of an embodiment of the system constructed in accordance with the invention;

FIG. 1(b) is a flow chart of an embodiment of the operation of the system of FIG. 1(a);

FIG. 2(a) is a screenshot of a patient top level display as shown on the user interface of FIG. 1, according to an embodiment of the invention;

FIG. 2(b) is a screenshot of the first page of a patient data entry display according to an embodiment of the invention;

FIG. 3 is a screenshot of a patient data display for entering the patient complaint for the present visit according to an embodiment of the invention;

FIG. 4 is screenshot of a patient body atlas data entry display according to an embodiment of the invention;

FIG. 5(a) is screenshot of a patient data entry display for entry of data on the body atlas according to an embodiment of the invention;

FIG. 5(b) is screenshot of a patient data entry display for entry of data on the body atlas according to an embodiment of the invention;

FIG. 6 is screenshot of a patient data entry display showing the data entry for the complaint listed in FIG. 3 according to an embodiment of the invention;

FIG. 7 is screenshot of a patient data display showing database results for drugs used by physicians for the treatment of the patient's diagnosis according to an embodiment of the invention;

FIG. 8 is screenshot of a patient data entry display for the entry of additional data on the body atlas of the patient according to an embodiment of the invention;

FIG. 9 is screenshot of a patient data entry display with the entry of photographic data according to an embodiment of the invention;

FIG. 10 is a screen shot of a patient procedure request page according to an embodiment of the invention;

FIG. 11 is screenshot of a patient summary data display according to an embodiment of the invention;

FIG. 12 is screenshot of a patient trend data display according to an embodiment of the invention;

FIG. 13 is screenshot of another patient summary data display according to an embodiment of the invention; and

FIG. 14 is a diagrammatic representation of the interaction of the data structures of an embodiment of the invention.

### DESCRIPTION OF AN EMBODIMENT OF THE INVENTION

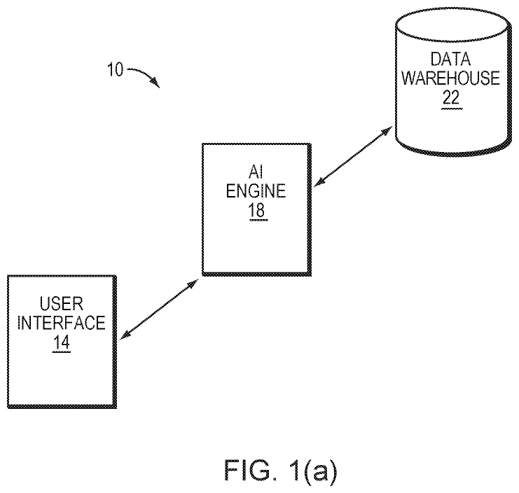

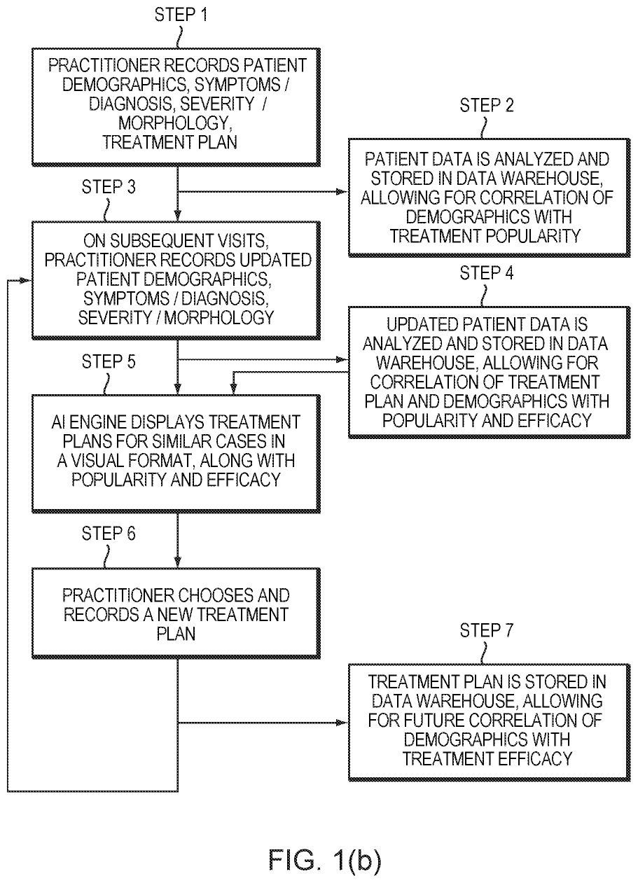

Referring to FIG. 1(a), the system 10 of the present invention includes a user interface 14 which communicates with a learning or AI engine 18. A physician or other medical healthcare provider (who is referred to herein as a physician without the loss of generality) provides patient data to the AI engine 18 and receives results through the user interface 14. The AI engine 18 analyzes the input data and stores the data in a data warehouse 22. Unlike systems that simply provide a template and store the information entered about a patient, the present system learns about the physician making the examination, such as medicines prescribed for a given diagnosis or procedures used, and provides those learned choices to the physician for incorporation into the patient record. In addition, the AI engine 18 mines the data warehouse 22 to provide calculated information to the doctor, such as patient physical trends and what other physicians are prescribing for the specific diagnosis. Thus, not only does the system have access to the patient's data in the data warehouse, but it has access to other patients' data concerning how the other patients have responded to a given medicine or procedure, as well as ancillary information such as whether the treatment was considered reimbursable for the purposes of health insurance coverage.

Referring to FIG. 1(b), a flowchart of the use of the system to select a treatment regime for the patient. The clinician records (Step 1) the patient's demographics (which may include but are not limited to: age, sex, race, preexisting conditions and procedures, and family history), symptoms, diagnosis, severity, morphology, and treatment plan. The patient's data is analyzed and stored in data warehouse, allowing for correlation of demographics with treatment popularity (Step 2). On subsequent visits, the clinician records (Step 3) updated patient demographics, symptoms, diagnosis, severity, and morphology. Again, this updated patient data is analyzed and stored (Step 4) in data warehouse, allowing for correlation of treatment plan and demographics with popularity and efficacy. The AI engine displays treatment plans (Step 5) for similar cases in a visual format, along with popularity and efficacy. The clinician can then choose and record a new treatment plan (Step 6). The new treatment plan is subsequently stored (Step 7) in the data warehouse, allowing for future correlation of demographics with treatment efficacy.

As this process is repeated for many patients and clinicians in the same practice and different practices, the system shows us a visual display of the diagnosis over time, plotting severity and morphology against different treatment plans, so the practitioner can spot trends. The AI engine/data mart compares demographic data and symptoms/diagnosis of current patient to other similar patients, giving a visual display of statistically similar cases and the treatment plans associated with those patients, and the efficacy of those treatment plans. The clinician uses these two visual displays to help determine a new treatment plan and the new treatment plan is recorded for future correlation of efficacy.

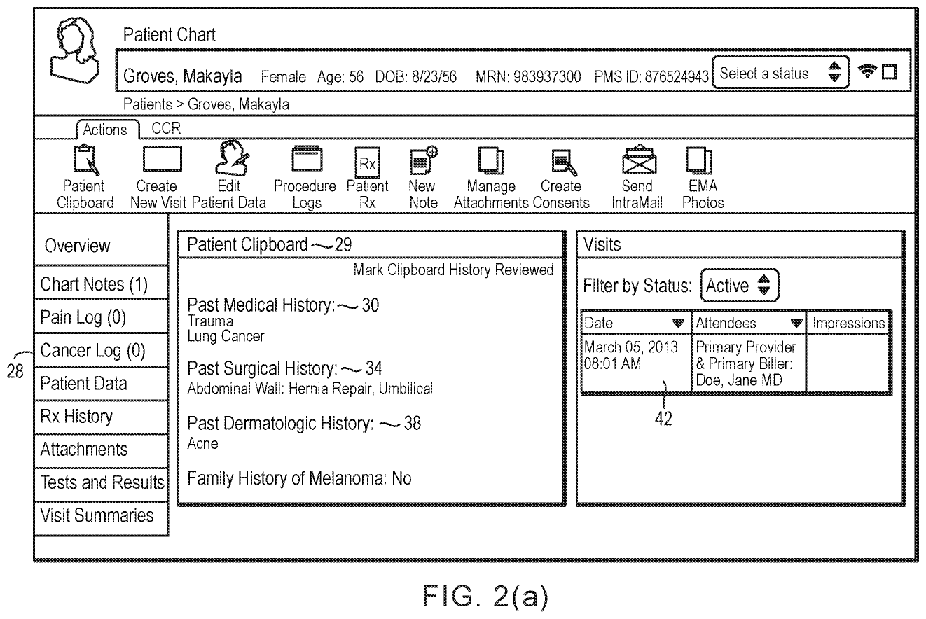

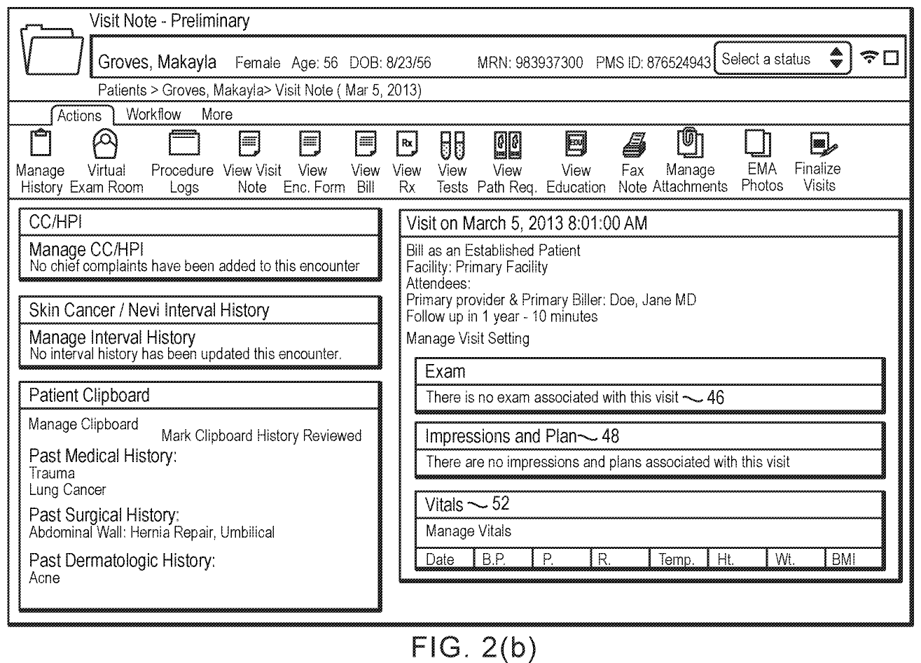

Referring to FIG. 2(a), the system 10 first provides an initial patient record that includes frame 28 listing available information and a “clipboard” frame 29 with patient medical 30, surgical 34, and dermatological 38, etc., histories. By selecting the current visit 42, the system 10 provides an input screen (FIG. 2(b)) that allows the physician to record examination notes 46, treatment plan 48 and vital signs 52.

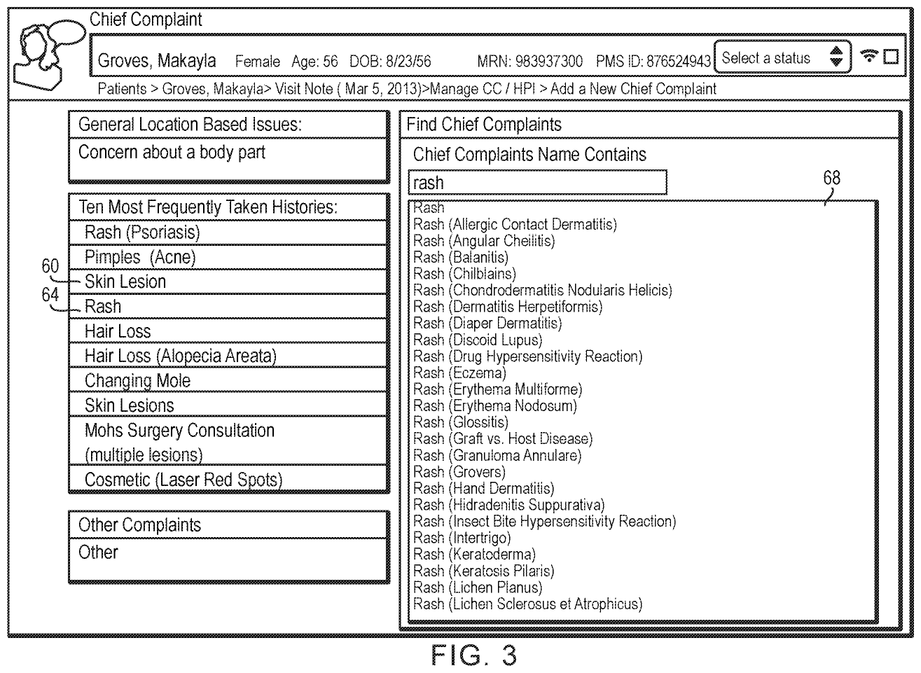

Referring to FIG. 3, to enter the patient's complaint, the system displays to most frequently seen complaints reported by the physician. By selecting “rash” 64, the system displays a pop-up menu 68 of various descriptions pertaining to rashes.

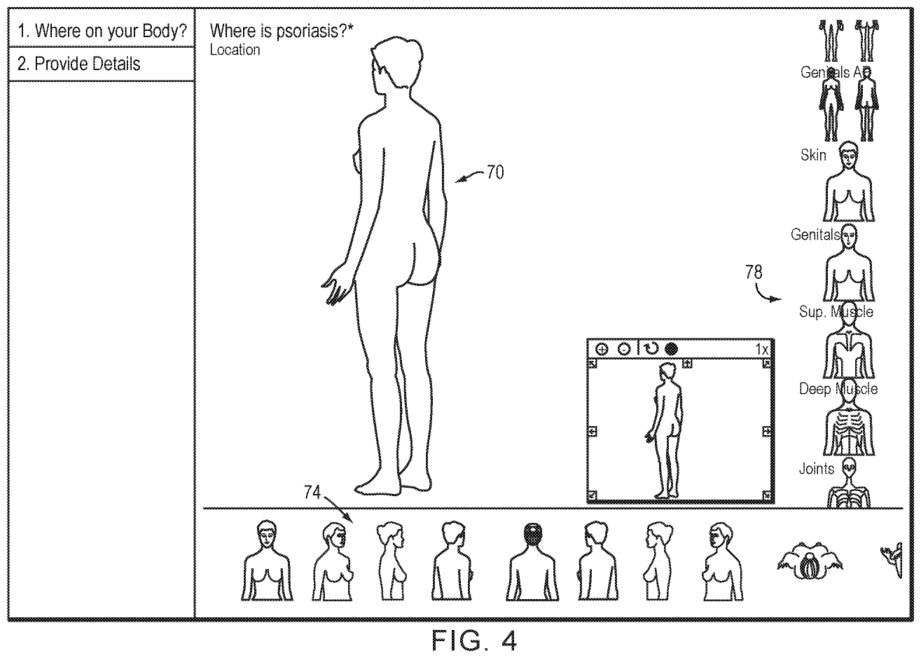

The next screen, (FIG. 4), shows a “body atlas” which provides an anatomical 3-dimensional diagram 70 of the patient. This patient's diagram may be rotated in 3-dimensions by selecting the desired orientation 74. In addition, the tissue level 78 can be selected, such as subcutaneous, muscular, and skeletal.

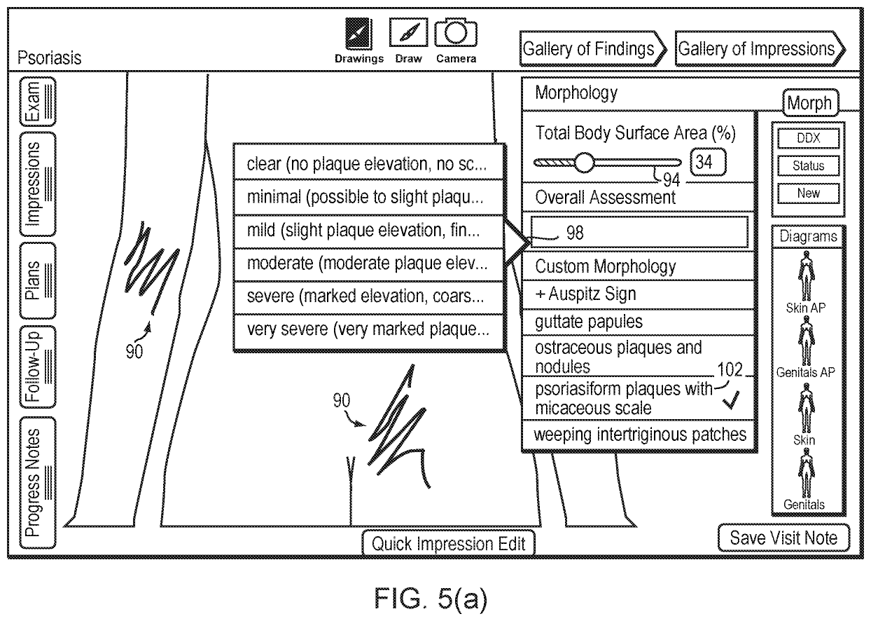

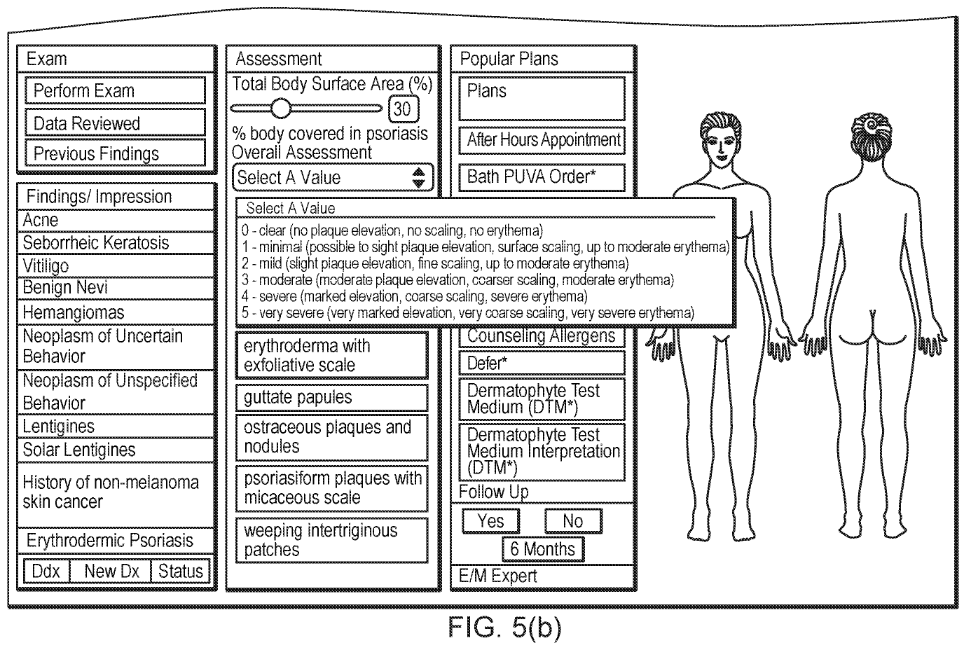

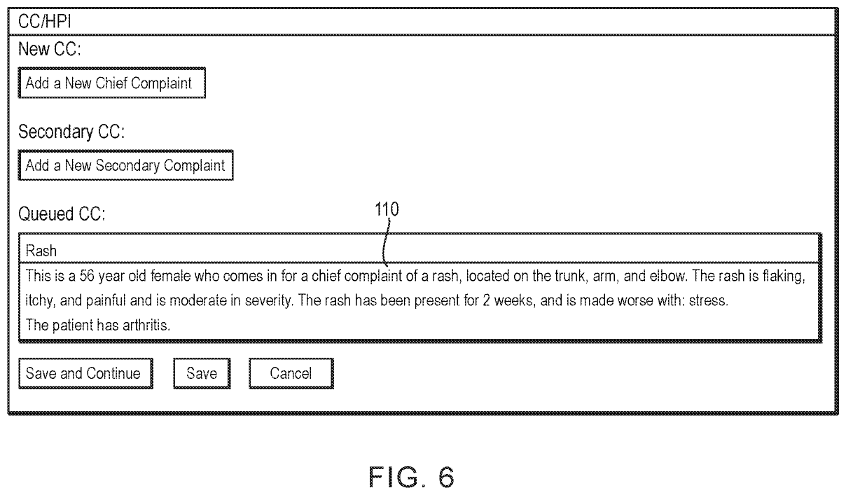

Referring to FIG. 5(a), a patient data entry screen is shown that allows the physician to enter notes using the patient atlas, including location 90 and, through dropdown menus, various parameters for patient assessment. In the view shown, this is being done for dermatological assessment, but other specialties are also possible. In addition to indicating where on the body the dermatological issues present 10, the physician can also indicate total body area covered 94, severity assessment 98, and morphology 102. Various embodiments of the interface (FIG. 5b) allow data to be entered through various dropdown menus. The system uses the information in the record to generate (FIG. 6) a written report 110 without requiring the physician to type anything.

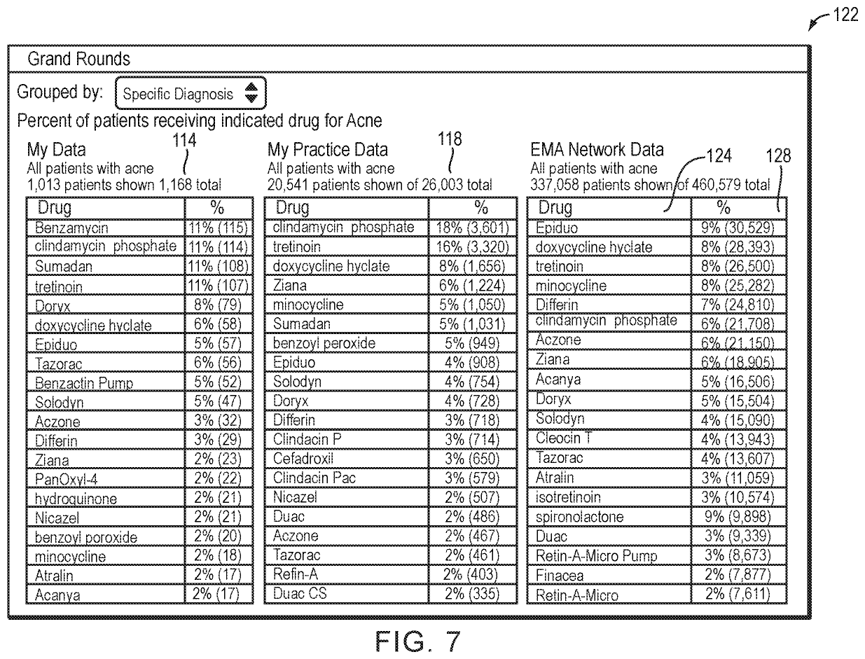

Referring to FIG. 7, it is possible for the clinician to interrogate the database to determine what percentage of the patients with a specific disease are being given what drug and whether that drug is being successful in treatment. The system 10 interrogates the data warehouse 22 and is capable of determining what the physician typically prescribes 114, what is prescribed by all the physicians in his practice 118, and what all physicians, using the system nationwide, use 122. In the embodiment shown, the drug is listed 124, as well as the total number of physicians prescribing the drug for that diagnosis and the percentage of physicians 128 prescribing the drug for that diagnosis. It is important to note that the data in the database is not limited to one physician's patients' data, but includes the data of all patients whose physicians utilize the system. The number of patients whose data is in the database can be millions.

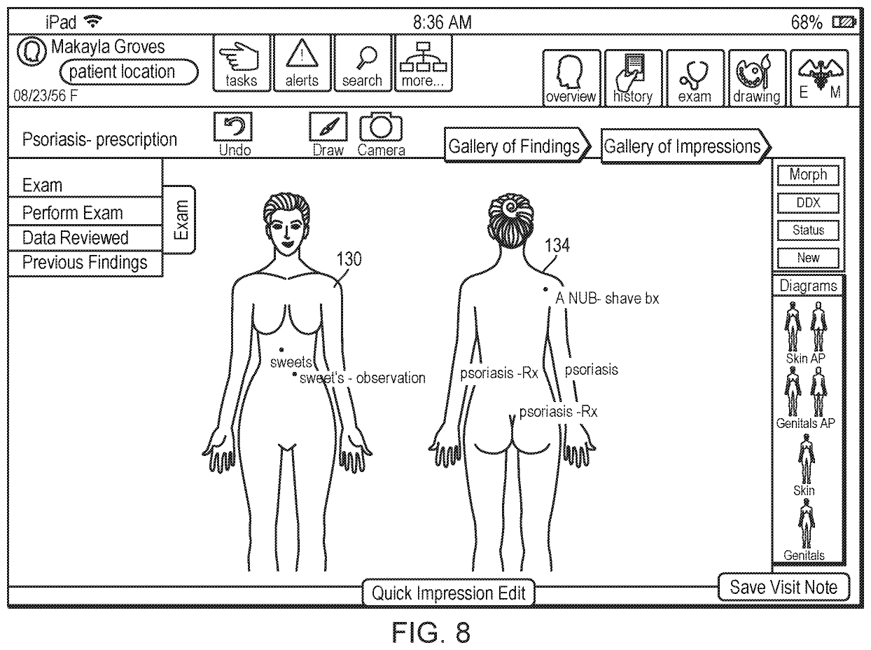

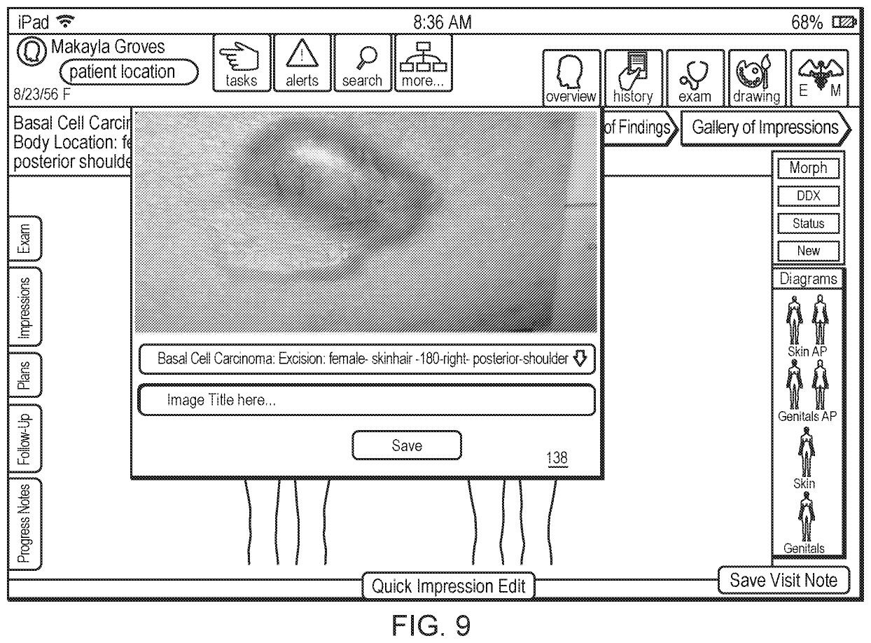

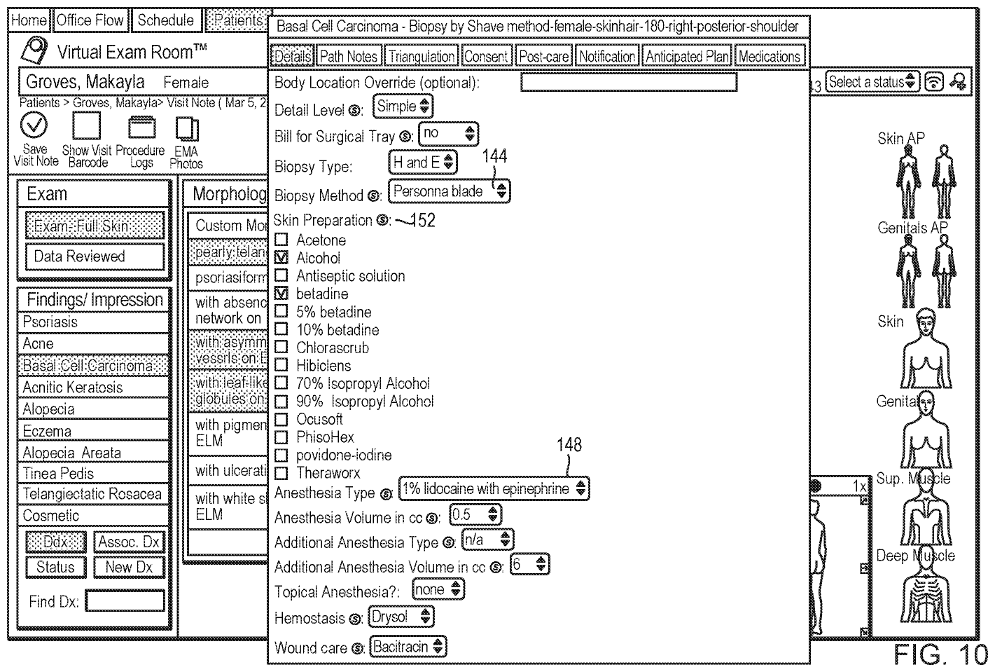

Referring to FIG. 8, the physician can then return to an earlier screen and indicate the other medical issues and their locations 130 (sweets syndrome) and 134 (basal cell carcinoma). The physician may attach a photograph 138 (FIG. 9) to a location 134 (FIG. 8). The physician can then designate what procedures are to be used to verify the diagnosis. Referring to FIG. 10, the system will pre-populate the procedure order, using what the physician has indicated previously that he or she prefers for this type of diagnosis, such as biopsy method 144, anesthesia 148, and skin preparation 152. The physician may also provide the instructions he or she prefers for pre-surgical preparation. These settings are “sticky” and are assumed to be the default settings going forward unless changed by the physician.

It is also not significant that the person entering the patient data may not be the physician. The system may be set to use any caregiver's preferences for any data being entered. Thus, a nurse entering notes for a physician can specify that the physician is entering the notes and that physician's verbiage and parameter preferences will appear on the screen. Thus, the knowledge database is linked to the specified provider.

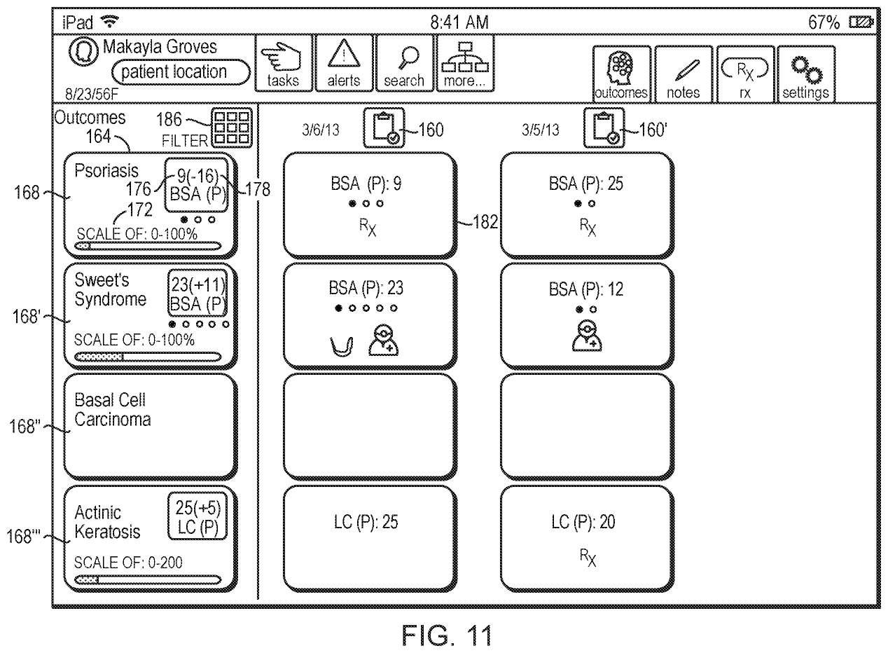

Referring to FIG. 11, the physician can request all information regarding the various diagnoses of a given patient displayed against visits 160, 160′ (generally 160). The fax left column 164 shows each diagnosis (generally 168), the severity 172 as measured by a specific scale 176, and how it compares to the last measurement 178. Specific complaints/diagnoses/outcomes are depicted as improving, regressing, or stable by correlating assessment measurements of severity across multiple visits. The outcome is depicted in such a way, using color and iconography, to make it easy to assess these conditions at a glance. By selecting a given visit and complaint 182, the clinician can see an entire chart in a standard format.

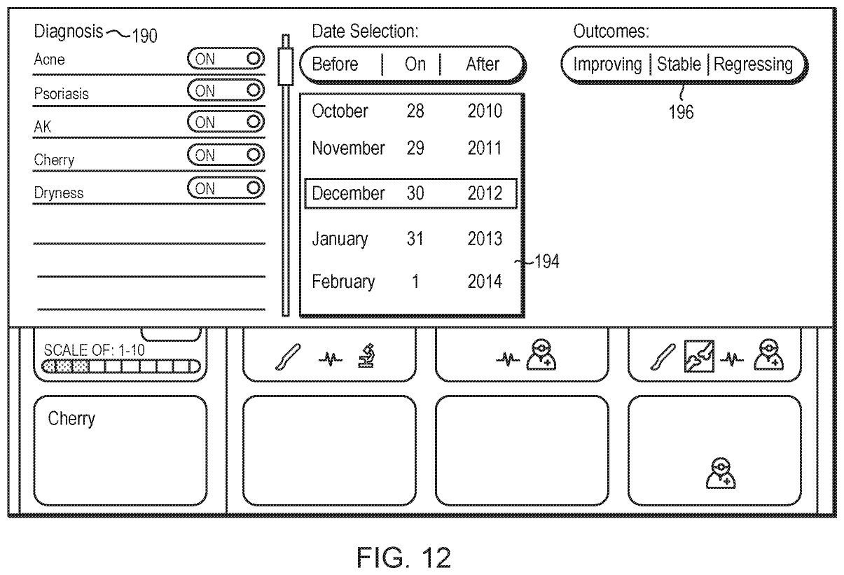

By selecting the filter icon 186 (FIG. 11), the physician can select filter criteria for viewing the data (FIG. 12). Thus, the data can be data subsets such as diagnosis 190, date 194, drugs used, co-morbidity, disease hierarchy, and so on. Filters can also include diagnosis outcome 196, such as improving, regressing or stable.

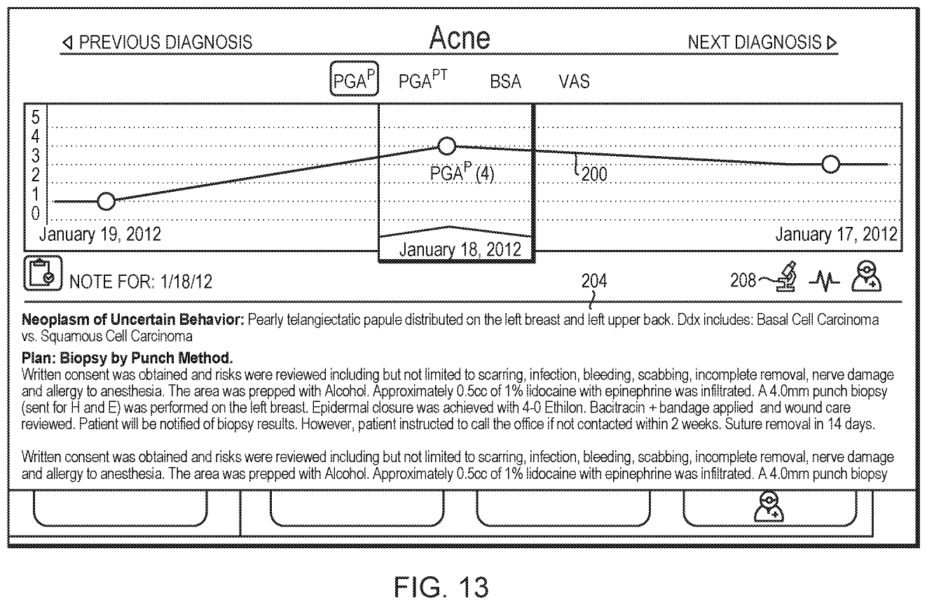

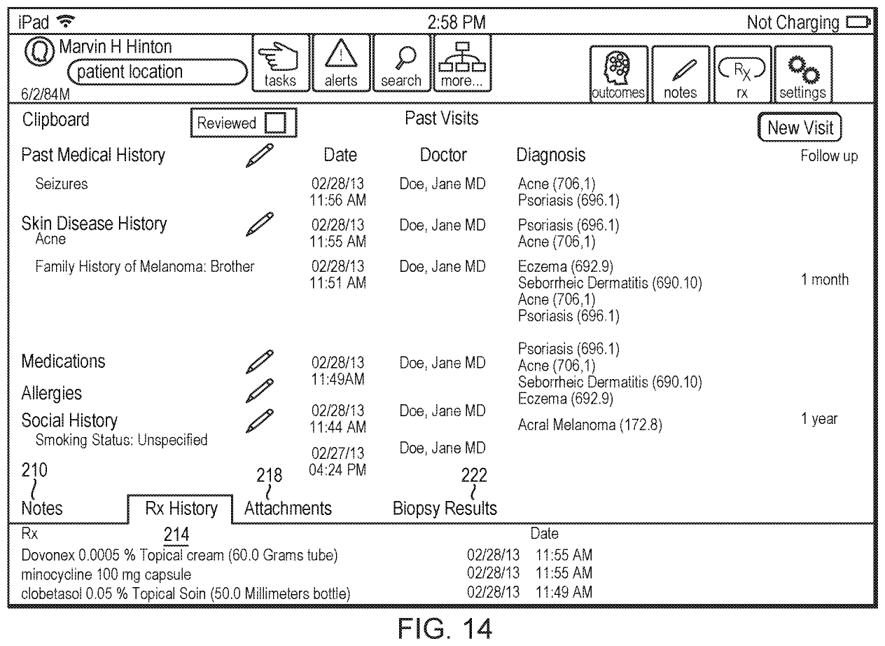

Alternatively, the clinician can select trending (FIG. 13), showing how the patient's assessment on a per-diagnosis basis is varying over the visits 200. In addition, the written record commentary is shown in a window 204, and the user can jump to various specific queries by selecting the icons 208. The system can also generate a summary (FIG. 14), including notes 210, prescription history 214, attachments 218 and biopsy results 222, by date of visit.

All this functionality is only possible because of the use of an intelligent learning engine in conjunction with a data warehouse. Data for the system are stored across multiple databases in the warehouse, which in one embodiment is in the network cloud. Identifiable patient data are accessible only by the patient's caregivers. However, de-identified data are aggregated into the data warehouse through a custom build extract, transform, and load (ETL) process. In this manner, data as to what drugs are being prescribed for what diagnoses become available (FIG. 7) without any loss of patient privacy. The database is therefore a multidimensional database whose data may be accessed through any combinations of dimensions. Filters are applied according to the parameters specified by the physician and to limit the dimensional space that must be searched.

The aggregated data are then summarized into a data mart. The data mart provides the data used by the application in a maximally summarized and indexed way, and all values that can be pre-calculated and pre joined are. This summarization in a data mart permits all queries issued by the application to be executed quickly.

As an example of a data mart, the physician, as described previously, can view the most common medications prescribed for a given diagnosis. Diagnoses in the database are aggregated, in one embodiment, at an atomic level available using a diagnosis name attribute of the diagnosis. Alternatively, the diagnoses are aggregated by their International Classification of Diseases, (generally ICD codes) or other code. Similarly, medications, in one embodiment, are aggregated by their FDB™ (First Databank, San Francisco, Calif.)/RxNorm (National Institutes of Health, Bethesda, Md.) drug name such that all doses, formulations, etc. that fall under a given drug name are included in a single grouping. Three of the data sets are: data showing what a given provider has used in the past for a given diagnosis; data showing what the providers in a given practice have used in the past for a given diagnosis; and data for the network showing what all of the providers using the system are prescribing. The system utilizes the information such as ICD codes and diagnostic codes to provide other ancillary information such as whether or not the costs associated with a given treatment are likely to be reimbursed by the health insurer. Further, because of the access to the enormous amount of data in the warehouse, the system can correlate and report upon the popularity, efficacy and the return on billing of the various drugs, procedures, and other treatments used in the treatment of the patient.

The data comprising physician preferences are stored in a database. Upon entry of the physician's name into the system during the patient visit session, the preferences are accessed by the system. A diagnosis entry then links to morphology entries, which link to the atlas through coordinates on the image. As a diagnosis is made, the preferences are linked to the required preferences.

It is to be understood that the figures and descriptions of the invention have been simplified to illustrate elements that are relevant for a clear understanding of the invention, while eliminating, for purposes of clarity, other elements. Those of ordinary skill in the art will recognize, however, that these and other elements may be desirable. However, because such elements are well known in the art, and because they do not facilitate a better understanding of the invention, a discussion of such elements is not provided herein. It should be appreciated that the figures are presented for illustrative purposes, and not as construction drawings. Omitted details and modifications or alternative embodiments are within the purview of persons of ordinary skill in the art.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A medical information system comprising:
a learning engine;
a user interface in communication with the learning engine, and
a data warehouse in communication with the learning engine,
wherein the learning engine will generate any of a number of reports relating to a current patient based in response to the current patient's diagnostic data, and current patient's demographics and patient data and demographics of other patients' data in the warehouse.

2. The medical information system of claim 1 wherein the data warehouse includes at least one data mart comprising summarized and indexed aggregated data that are pre-calculated and pre-joined.

3. The medical information system of claim 1 wherein the generated report comprises one or more of a measure of the popularity and a measure of efficacy of treatment and a determination of the amount of reimbursement on billing.

4. The medical information system of claim 2 wherein medicines are aggregated by their FDB/RxNorm drug name.

5. The medical information system of claim 2 wherein the learning engine will filter requests for aggregated data based on parameters supplied by the user interface.

6. The medical information system of claim 2, wherein a user may query medicine treatment statistics based in response to diagnostics and the patient's data and aggregate data in the data warehouse.

7. The medical information system of claim 1 wherein some of the plurality of preferences of the physician are predetermined by selection by the physician.

8. The medical information system of claim 1 wherein some of the plurality of preferences of the physician are predetermined by actions taken by the physician.

9. The medical information system of claim 1 wherein the learning engine further comprises an interactive 3-D body atlas comprising a plurality of tissue levels, wherein the physician may annotate the body atlas with patient data.

10. The medical information system of claim 9, wherein the annotated body atlas with patient data is linked to other aggregated data in the database.

11. The medical information system of claim 1 wherein the learning engine will generate a report on a patient based in response to a plurality of preferences of the physician attending to the patient.

12. The medical information system of claim 1 wherein patient trend data is displayed on the user interface.

13. A method of using a medical information system, the medical information system comprising: a learning engine; a user interface in communication with the learning engine; and a data warehouse in communication with the learning engine, comprising the step of generating a report on a patient by the learning engine generated in response to patient data.

14. The method of claim 13 further comprising the step of summarizing and indexing aggregated data that are pre-calculated and pre-joined in at least one data mart.

15. The method of claim 14 further comprising the step of filtering requests for aggregated data based on parameters supplied by the user interface.
